(19) United States
(12) Patent Application Publication
(10) Pub. No.: US 2012/0289896 A1
(43) Pub. Date: Nov. 15, 2012

(54) PERCUTANEOUSLY DEPLOYED ABDOMINAL DRAIN

(76) Inventors: Timothy R. Wolfe, Salt Lake City, UT (US); Marshall T. Denton, Salt Lake City, UT (US); J. Michael Brown, Salt Lake City, UT (US); Mark A. Christensen, Salt Lake City, UT (US); Edward J. Kimball, Salt Lake City, UT (US); Michael L. Cheatham, Orlando, FL (US); Huy N. Tran, Riverton, UT (US); Perry W. Croll, Salt Lake City, UT (US)

(21) Appl. No.: 13/470,107

(22) Filed: May 11, 2012

Related U.S. Application Data
(60) Provisional application No. 61/518,920, filed on May 12, 2011.

(51) Int. Cl. A61M 1/00; A61M 5/14

(52) U.S. Cl. 604/43; 604/540

(57) ABSTRACT

Devices adapted to facilitate draining fluid from the abdominal/peritoneal cavity of a medical patient. The invention may be embodied in one or more element of an access port, insertion assist device, and/or abdominal catheter. A preferred abdominal catheter provides a drain field that can be inserted into the abdominal compartment, through an access opening having a small cross-section, in a stowed configuration and subsequently expanded to provide a large drain area through which to extract fluid from the compartment.

### CROSS-REFERENCE TO RELATED APPLICATION

This application claims the benefit of U.S. Provisional Patent Application Ser. No. 61/518,920, filed May 12, 2011, the disclosure of which is hereby incorporated herein in its entirety by this reference.

### TECHNICAL FIELD

The invention relates to devices adapted to remove fluid from an interior portion of a medical patient. In particular, certain embodiments are adapted to remove fluids from the peritoneal cavity/abdominal compartment of a human medical patient. Some embodiments may also permit infusion of fluid into that cavity or compartment, as well as direct measurement of intra-abdominal pressure.

### BACKGROUND

Elevated intra-abdominal pressure leads to major changes in the body's physiology that, if undetected and untreated, can result in organ damage and patient death. When patients become critically ill, they may develop a capillary leak phenomenon that causes the tissues in their body to become edematous with extra fluid that seeps out of the capillaries. This process is called “3rd spacing” of fluid. The condition is very common in sepsis, burn, trauma and post-operative patients. One area of the body where 3rd spacing is especially prevalent is the abdominal cavity. Critically ill patients can have many liters of fluid leak into the intestinal wall, the intestinal mesentery, and the abdominal cavity (as free fluid sloshing around the intestines).

Fluid 3rd spacing in the abdominal cavity results in an increase in intra-abdominal pressure (IAP). Normal IAP is 0 mm Hg to subatmospheric (less than 0 psig). Once the pressure builds to 12-15 mm Hg, intra-abdominal hypertension (IAH) occurs. At this point, methods to improve intestinal perfusion should be started, such as: fluid loading to increase blood flow to gut, inotropic support to increase cardiac output, etc. As pressures increase above 20-25 mm Hg, the abdominal compartment syndrome (ACS) exists and major physiologic and organ system dysfunction result. Decompressive surgery (vertical midline abdominal incision) is often required to prevent irreversible organ damage and death. The exact pressure at which abdominal decompression should occur is dependent on a number of host factors including age, underlying co-morbidities and physiologic evidence of developing ACS.

Decompressive surgery is an aggressive treatment of last resort. Fluid-engorged viscera occupy a larger volume than the abdominal compartment provides, and portions essentially spring out from the abdominal compartment through the emergency abdominal incision. Often, some sort of temporary external covering is required to provide protection to viscera that extend from the abdominal compartment during the time interval while excess fluid is removed, and visceral volume is reduced to a conventional size. It is believed preferable to begin treatment at greatly reduced IAH levels. One treatment method within contemplation includes insertion of one or more catheter into the abdominal compartment to drain 3rd-spaced fluids.

Cytokines are present during onset of IAH. It is believed that cytokines participate in, and may even be a cause of, increased IAP. A workable treatment to reduce IAP may include infusion of fluids into the abdominal compartment to dilute and remove cytokines.

An exemplary known catheter structured to drain fluid from a cavity of a medical patient includes the curved drainage catheter included in the Percutaneous Cavity Drainage Catheterization Kit currently available under part No. AK-01600 from TELEFLEX® at world wide web address arrowintl.com. Such a catheter has a smooth cylindrical body with an insertable length of about 9 inches (229 mm); a diameter of about 0.18 inch (4.6 mm); a cross-section area of about 0.0143 in² (9.2 mm²); and caries 3 approximately oval body-wall apertures, each aperture having a major diameter of about 0.25 inch (0.6 mm) and a minor diameter of about 0.125 inch (0.3 mm), producing an open aperture area of about 0.024 in² (15.8 mm²). Such catheter provides a total aperture area of about 0.074 in² (47.5 mm²).

### DISCLOSURE

The invention may be embodied in one or more devices adapted to facilitate removal of fluid from an abdominal compartment of a medical patient. One exemplary such device may be characterized as an access port that facilitates access to the abdominal cavity through the body wall of a medical patient. An access port may non-exclusively include structure configured to: resist undesired escape of fluid from the compartment; resist undesired removal of the port from an installed position; facilitate installation of a draining catheter; and/or temporarily close a compartment access opening.

Another device within the ambit of certain principles of the instant invention may be characterized as an insertion assist device. An exemplary insertion assist device may non-exclusively include structure configured to: retain a drain device in a stowed configuration; dilate an access tunnel through a body wall; permit transverse extraction of the device from a mid-span location of a drain umbilical; facilitate passage of a drain device through the body wall; and/or guide a drain device toward a desired installation position inside an abdominal compartment.

Another device within the ambit of certain principles of the instant invention may be characterized as an abdominal catheter. An exemplary such catheter may non-exclusively include structure configured to: reduce in size to form a compact, stowed configuration; resist undesired escape of fluid from the abdominal compartment; resist undesired removal of the catheter from an installed position; facilitate installation of a distal portion of the catheter into an abdominal compartment; deploy from a stowed configuration to dispose a drain field in an expanded configuration; deploy from a stowed configuration to act as a bridge element associated with a drain aperture; apply infusion fluid to an abdominal compartment; directly measure pressure in the abdominal compartment; and/or cause an enhanced active drain area.

An abdominal catheter according to certain principles of this invention provides a drain field, which can be deployed inside the abdominal compartment of a medical patient through a small-diameter access opening in the patient's body wall and extract fluid from that compartment. One exemplary such drain field may include an envelope that is deployed from a stowed configuration to a deployed configuration. Envelope deployment mechanisms within contemplation include mechanical linkage elements and inflatable elements. An exemplary envelope may be urged from a stowed configuration toward a desired deployed configuration by inflation of a skeleton. An inflatable member may enhance the active area of a drain aperture. Sometimes, an inflatable member may expand a crenellated surface for contact with the viscera. Sometimes, an inflatable member may cooperate with an aperture to space viscera way from the aperture and increase the effective active drain area. Inflation may encompass application of pressurized fluid or gas.

### BRIEF DESCRIPTION OF DRAWINGS

In the drawings, which illustrate what are currently regarded as the best modes for carrying out the invention:

FIG. 1 is a cross-section view in perspective of an access tunnel and installed access port;

FIG. 2 is a side view, partially in cross-section illustrating alternative structure of alternative workable access ports;

FIG. 3 is a side view in cross-section of another alternative access port;

FIG. 4 is a view in perspective, partially in cross-section, of a drain field at an initial state of being installed;

FIG. 5 illustrates a further state of installation of the embodiment in FIG. 4

FIG. 6 is a side view illustrating an initial state of deployment of an alternatively structured drain field;

FIG. 7 is a side view illustrating final deployment of the embodiment in FIG. 6;

FIG. 8 is a side view of a drain field embodiment according to certain principles of the invention;

FIG. 9 is a top view of the embodiment illustrated in FIG. 8;

FIG. 10 is a cross section taken through section indicated at 10-10 in FIG. 9;

FIG. 11 is a cross section taken through section indicated at 11-11 in FIG. 9;

FIG. 12 is a cross-section view, similar to that shown in FIG. 4, of an alternatively structured drain field embodiment, with deployment structure in an inflated configuration;

FIG. 13 is a cross-section view of the embodiment of FIG. 12, but with the deployment structure deflated;

FIG. 14 is a cross-section view of an alternative drain field;

FIG. 14A is a cross-section view of an alternative drain field;

FIG. 15 is a top view of the embodiment illustrated in FIG. 14;

FIG. 15A is a top view of the embodiment illustrated in FIG. 14A;

FIG. 16 is a cross-section view of the embodiment illustrated in FIG. 14, with deployment structure in an inflated configuration;

FIG. 16A is the cross-section view of the embodiment illustrated in FIG. 15A, indicated by section 16A-16A, with deployment structure in an inflated configuration;

FIG. 17 is a cross-section view of the embodiment illustrated in FIG. 14, with deployment structure in a deflated configuration;

FIG. 17A is the cross-section view of the embodiment illustrated in FIG. 15A, indicated by section 17A-17A, with deployment structure in a deflated configuration;

FIG. 18 is a side view of a workable insertion-assist tool; and

FIG. 19 is a side view of an alternative insertion-assist tool;

FIG. 20 is a side view of a portion of a multi-lumen conduit that may be used in certain embodiments structured according to certain principles of the invention;

FIG. 21 is a side view of an exemplary arrangement effective to facilitate installation of a drain field assembly;

FIG. 22 is an alternative arrangement to effect drain field installation;

FIG. 23 is a view in perspective of a currently preferred abdominal drain assembly;

FIG. 24 is a side view of an abdominal catheter similar to the catheter in FIG. 23;

FIG. 25 is a top view of the abdominal catheter in FIG. 24;

FIG. 26 is a close-up cross section view of the tip of the catheter in FIG. 24;

FIG. 27 is an exploded assembly view in perspective looking at the distal end of the catheter in FIG. 24;

FIG. 28 is a view in perspective looking at the distal end of an alternative abdominal catheter;

FIG. 29 is a side view of the catheter in FIG. 28;

FIG. 30 is a distal end view of an alternative abdominal catheter;

FIG. 31 is a view in perspective of a portion of the catheter in FIG. 30

FIG. 32 is a cross-section view of the catheter in FIG. 33, taken through section 32-32;

FIG. 33 is a side view of the catheter in FIG. 30;

FIG. 34 is a close-up cross-section view in perspective of a hub portion of the catheter in FIG. 30;

FIG. 35 is a side view of a partially assembled alternative abdominal catheter;

FIG. 36 is a cross-section view of the tip end of the catheter in FIG. 35; and

FIG. 37 is an end view of the catheter in FIG. 35.

### DETAILED DESCRIPTION OF THE INVENTION

The present invention provides various apparatus and methods for draining fluid from an interior portion of a medical patient. In particular, currently preferred embodiments structured according to certain aspects of the invention are adapted to facilitate draining fluid from the abdominal compartment of a medical patient. Such treatment may be administered, for non-limiting examples, as a portion of treatment for symptoms of intra-abdominal hypertension, or to guard against such malaise, or to facilitate a dialysis or dilution procedure. It is within contemplation that embodiments structured according to certain principles of the invention may be scaled in size, and find application in connection with treatment of other areas of a medical patient.

One currently preferred method for treating a medical patient for elevated abdominal compartment pressure incorporates an access opening formed through the patient's abdominal wall. Desirably, the access opening is sufficiently small as to avoid need for complete dissection to apply closing sutures to reclose the opening through the fascia. Therefore, it is preferred to make an opening through the fascia that is small enough to resist formation of hernias through the fascia by internal organs. A currently preferred opening in the fascia is less than about 12 mm in a measured characteristic size, such as diameter. A more preferred opening size is less than about 10 mm. The currently most preferred opening size is less than about 8 mm.

Desirably, a drain device according to certain principles of the instant invention is adapted for percutaneous deployment. An access tunnel into the abdomen may be effected by way of needle puncture through the skin and body wall. In an alternative deployment, a drain field structured according to certain principles of the invention may sometimes be surgically implanted. In any case, drain fields structured according to certain principles of the invention are desirably removable from a patient's cavity through an aperture that is small enough to avoid need for complete dissection to apply closing sutures to reclose the aperture through the fascia.

A workable opening, or access tunnel, through a body wall may be formed using the Seldinger technique. In accordance with such well-known technique, the patient is typically disposed on their side to help gravity pull viscera away from the tip of a needle that is inserted into the abdominal cavity. Once the fascia is penetrated, a guide wire may be fed through the needle and into the peritoneal cavity. The guide wire typically remains in place for at least the subsequent dilation procedure. The skin at the puncture site may be incised to facilitate access, and a first dilator slid along the guide wire to enlarge the opening, which extends through the remaining body wall and fascia, by a controlled amount. Increasingly larger-size dilators may then be slid along the guide wire to dilate the access opening, as desired. In one preferred method of deployment, the guide wire also remains in place during at least a portion of the deployment of a drain field.

If desired, an optional access port, generally 100, can be inserted into the access tunnel 102 through the patient body wall 104 using an operable technique. Among other characteristics, an access port may be structured to provide one or more of: a means to expand the access tunnel; a smooth and slippery access tunnel wall to facilitate drain field installation; a temporary storage container for a drain field prior to deployment, an installation-assist device; a fluid leak-resistant tunnel seal, which may include structure arranged to resist a leak path internal to (through) and/or external (along) the access port; an internal and/or external anchor to resist ejection from an installed position; and/or a length adjustability to accommodate body walls of different thicknesses.

In some cases, a distal portion of an access port may be installed by sliding it over a dilator. It is within contemplation that distal structure of an access port may be installed using alternative guide structure. For example, an insertion-assist tool may include a distal portion resembling a hollow cylinder. The distal end of the hollow cylinder may be inserted through the patient's body wall to dispose a discharge opening of the tool in operable position inside the abdominal compartment. Then, a distal portion of an access port, or a drain field, may be ejected from confinement inside of the insertion tool cylinder.

Desirably, an access port 100 is structured to resist its inadvertent ejection from an installed position, and also to permit extraction of the access port through the desirably small access opening subsequent to a period of treatment of the patient. It is within contemplation that an exterior flange 106 may simply be sutured, generally 108, to the patient's skin 110. However, currently preferred port ejection-resisting structures include an enlarged area, such as internal flange 112, configured for disposition inside the abdominal compartment and adjacent the desirably small-sized access opening in the fascia. One operable structure effective to form an enlarged area includes internal flange 112 that is typically oriented substantially transverse to an axis through the port-tube. Such a flange 112 may be either continuous or interrupted around a flange circumference.

Alternatively, an inflatable structure, generally 114, may be configured to form an operable enlarged area 112. For example, a toroidal balloon 116 carried at the distal end of a port-tube 118 may be inflated subsequent to installation of the port-tube. In FIG. 3, the alternative embodiment, generally 119, includes an inflation lumen 120 communicating from inflatable balloon 116 to a convenient and externally disposed connector, such as luer-locking connector 122. It should be noted that an access tunnel 100 may include a removable cap element to temporarily close the access opening, e.g. in the case where an abdominal catheter is removed, but the patient may still require additional future catheterization.

With reference to FIG. 1, it is sometimes desirable for an access port 100 to include structure arranged to resist undesired leaking of fluid from inside the abdominal compartment. Certain of such sealing structures may include structure operable to seal sufficiently against the compartment fascia as to resist leakage of fluid in an external direction along the outside surface of the port-tube. For example, an interior flange 112 may be structured to permit engagement of an area portion of the interior flange against the inside of the peritoneal cavity fascia, generally indicated at 124. Fascia-sealing structures within contemplation nonexclusively include: a deformable flange, including a self-biased flange; and an inflatable flange or collar; or a balloon.

Sometimes, external structure may be included to cooperate with internal structure to effect a clamping action through-the-thickness of the body wall. For example, an exterior flange may be biased in some way toward an interior flange. With reference to FIG. 2, it is within contemplation that an exterior flange 126 may interface with one-way ratchet teeth, generally 128 carried by the port-tube 118. Alternatively, the interface may be threads, generally 130. Other alternative interface structure will be apparent to one of ordinary skill in the art. It is within contemplation that the port-tube 118 may be trimmed to a desired length subsequent to its installation, thereby accommodating a plurality of patients, each such patient having a different body wall thickness.

With reference again to FIG. 1, an access port 100 may include tube seal structure, generally 132, arranged to resist passage of fluid through the interior of the access port-tube. Desirably, such tube-seal structure will also accommodate to, and fluidly seal against, elements and structures that pass through the access port. Exemplary tubular elements expected to pass through the access port include one or more substantially cylindrical fluid-carrying conduit. Operable tube-seal structures 132 include valve arrangements, such as flap valves, and dilating membranes. It is within contemplation to simply inject a sealing element, such as sterile silicone sealant. In the latter case, a backer element, such as a piece of cotton or gauze, may first be inserted into the port-tube to provide a back-pressure in the subsequently injected silicone effective to fill the voids between the port-tube wall and fluid conduit(s). However, it is alternatively within contemplation that an access opening may simply be “plugged” with something, such as cotton and/or gauze. In such case, a topical dressing is typically monitored, and changed as necessary. Alternatively, one or more sutures may be applied to the skin 110 to form a sphincter effective to place the externally opening end of tunnel 102 in sealing contact against penetrating drain tubes, or other structure.

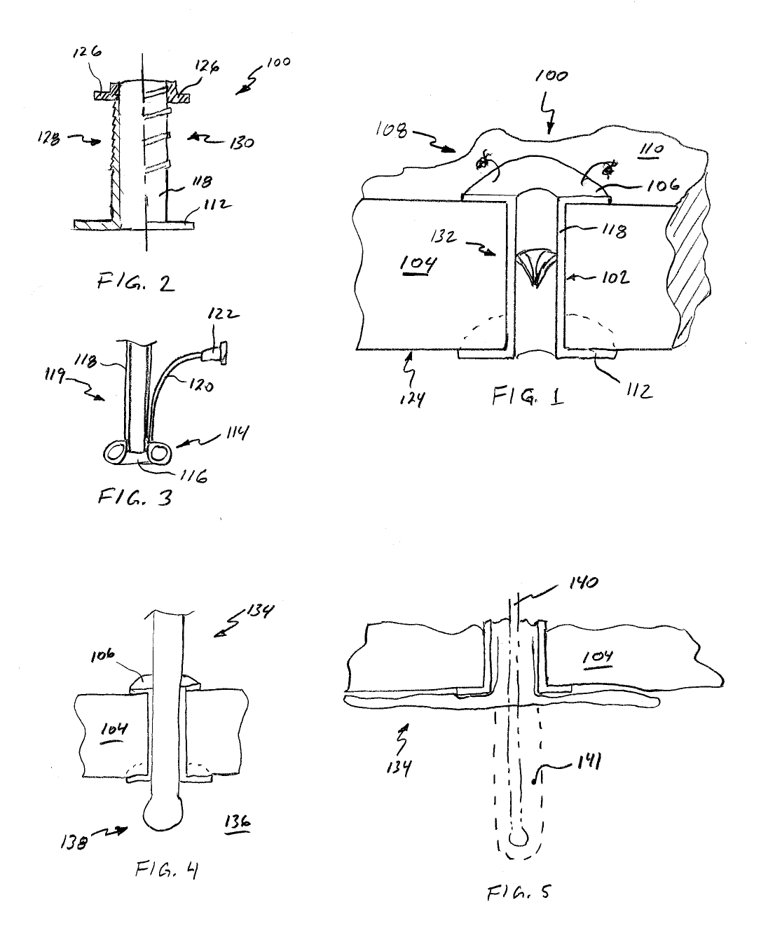

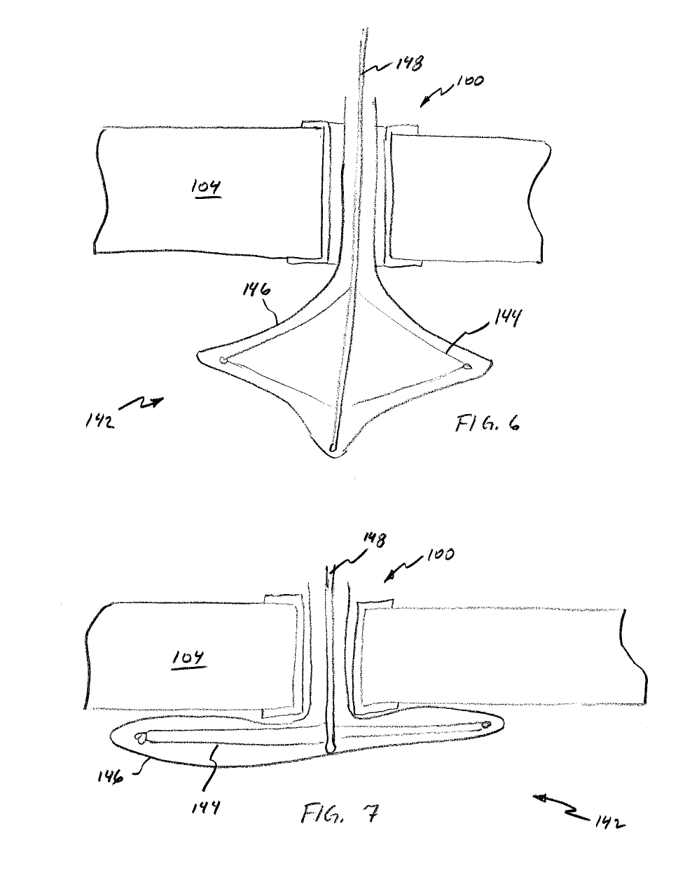

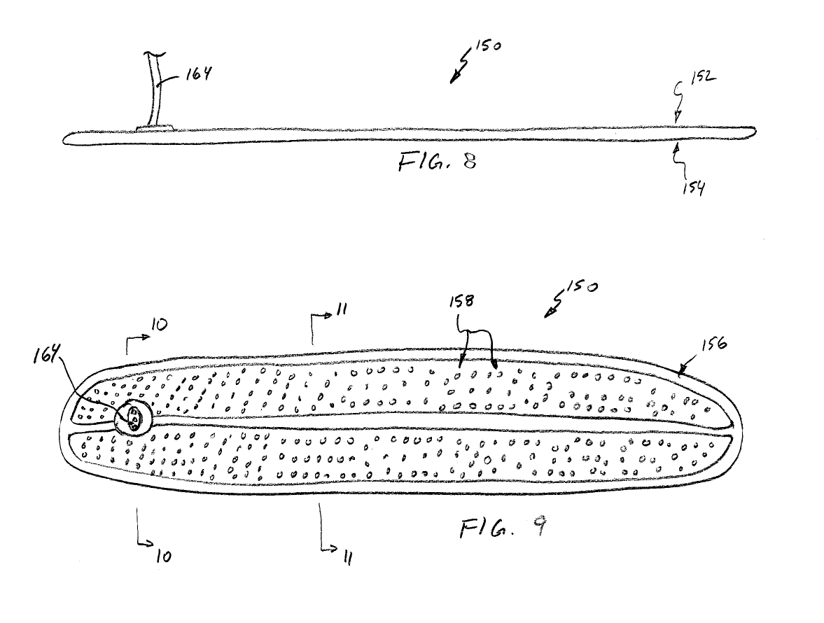

Once an access opening or tunnel 102 is established, an optional access port, such as previously described, may be installed. With reference now to FIGS. 4 and 5, a first embodiment of a drain field, generally 134, can then be inserted through the access opening in abdominal wall 104 and into the patient's abdominal compartment 136. Desirably, a drain field will include a blunt distal tip 138 to push on, without inflicting damage to, the viscera during installation of the drain field. In FIG. 5, the envelope indicated by phantom line shows an inserted position, prior to removal of installation rod 140. The embodiment 134 is biased to then inherently urge its configuration toward the solid line representation. Deployment may be triggered by the inserted device reaching body temperature, and may be effected by NITINOL™ wire, or other substance that returns to a pre-defined shape responsive to a temperature change. Subsequent to deployment, a deployed drain field is desirably removable through the access opening, as well. Sometimes, the interior 141 of a drain field may include fluid-flow-enhancing structure, such as ribs, pillars, walls, and the like, to resist collapse of the deployed drain field and facilitate flow of fluid toward a drain conduit and away from compartment 136. Typical dwell time for installation of a drain field in a compartment 136 is anticipated to be up to about three days before the drain field is removed from the patient. However, the dwell time increment is not a critical part of the invention.

A drain field may be defined as structure configured for insertion into an internal compartment of a patient and effective to convey fluid from the compartment toward a drain conduit. Typically, a drain field will include a plurality of relatively small apertures spaced apart by structure that provides one or more fluid flow path internal to the drain field and toward a fluid receptacle. Exemplary drain fields include exterior perimeter surfaces of a sponge, and a catheter having a plurality of apertures disposed along its insertable length. Apertures of a drain field structured for insertion into the peritoneal compartment are desirably sized less than about 3 mm in diameter (or other minimum characteristic size) to resist tissue in-growth or invasion. A minimum aperture size is effective to resist fouling, or “clogging” the aperture during its working deployment. It is currently preferred for drain apertures to be between about 1 mm and about 3 mm in diameter. In any case, a drain field is defined for purpose of this document as providing a composite draining surface area including a plurality of apertures through which fluid can pass from the compartment in which the drain field is disposed and into the flow path toward a fluid receptacle.

Desirably, a drain field is deployable to dispose a relatively large active draining surface area inside a body cavity or compartment. For purpose of this disclosure, and in the context of a drain field, the term “deployable” is intended to mean the drain field may be configured to have a first cross-section (or shape) prior to deployment of the drain field. (For example, a first cross-section may be defined by the intersection between the drain field and a plane disposed normal to a length axis, or an insertion axis, of the drain field). That first cross-section may be inserted through an aperture having a first size, and then undergo a change in the first cross-section during a deployment procedure to form a second cross-section that is distinguishable from the first cross-section.

In certain cases, a deployed drain field disposes a plurality of apertures in a different configuration in space compared to a pre-deployed configuration. In contrast, the active drain field portion of a conventional urinary catheter is not changed in configuration in space by the expansion of its retention balloon.

In other cases, a deployed drain field disposes a plurality of bridge and aperture pairs inside a compartment of a medical patient, with each such pair consisting of a different bridge and different aperture being operable in harmony to resist occlusion of each aperture of a respective pair by tissue inside the compartment. In contrast, a conventional urinary catheter includes, at most, one bridge and aperture pair, if the retention balloon can be regarded as a bridge. However, the balloon of a conventional urinary catheter is not a bridge, because the inflated balloon would not reasonably protect a distally disposed, and axially spaced apart, side-wall aperture from occlusion by tissue in a peritoneal compartment. Tissues internal to a peritoneal compartment would naturally slump and conform to the axial profile of a conventional urinary catheter in the vicinity of, and distal to, the balloon, thereby contacting the side-wall of the catheter in the vicinity of, and occluding, the side-wall aperture. For purpose of this document, a bridge must provide structure in association with an aperture effective to resist such occlusion and further space viscera from an aperture.

An exemplary bridge structure includes blocking structure disposed radially apart from the plane of, and proximal to, if not actually coincident with or covering, at least a portion of the associated aperture. Such an arrangement enforces a transfer space between viscera tissue and passive area of a drain field, through which transfer space fluid can migrate toward the aperture. The bridge also provides an enlarged contact gap through which fluid can be inspired from the viscera, as compared only to the open area of the aperture. Consequently, a “bridge” according to this document causes an increased active drain field area, compared to the aperture area, and causes a correspondingly bigger area on which suction may reliably be applied to the viscera. A workable bridge may be provided by crenellations, or even a balloon having a preferred configuration.

Also for purpose of this disclosure, the term “active” when used as a modifier for draining surface area is intended to mean an area that is realistically effective to extract fluid from an internal compartment of a medical patient. One such area is the composite sum total of cross-section areas provided by a plurality of apertures that are carried on a smooth surface, such as on the cylindrical surface of a known tubular drain catheter, such as the aforementioned curved drainage catheter. The entire installed cylindrical length of such catheter is not regarded as a portion of an “active” drain field, because the smooth wall portion is easily occluded by contact with anything, and consequently resists fluid movement toward an aperture. The smooth surface between apertures is regarded as providing a passive contribution to a total composite drain field area defined, in part, by the inserted length of such a catheter.

However, in the case of a crenellated surface having dispersed drain orifices communicating there-through to a suction source, essentially the entire surface area of the crenellated drain material may be regarded as “active,” in that the crenellations may provide a myriad of flow paths (transfer space) that resist occlusion and permit fluid flow from across essentially the entire crenellated surface area toward the drain apertures and then on toward a suction source or drained fluid receptacle. In general, a crenellated surface provides surface roughness (such as a series of bumps, columns, or walls), that spaces tissue away from transfer space in which fluid may migrate toward a drain path. A sponge may provide a similar uneven perimeter surface area that is essentially entirely active.

When the access opening through the fascia is of the desirably small size (e.g., 8 mm in diameter, or less), the drain field is generally configured to expand in at least one dimension, to form a desirably large deployed drain area. Such deployable drain fields may sometimes be made reference to in this disclosure as expandable drain fields, although such may also sometimes be adduced in context. In any event, a drain field structured according to certain aspects of the instant invention provides a drain field that has a first configuration permitting installation of the drain field into a cavity or compartment, and is subsequently “deployed,” or expanded, to have a second configuration. In contrast, known drain fields having a suitably large size require surgical placement, and a correspondingly large access opening.

Embodiments of expandable drain fields may sometimes include one, two, three, or even four lumen that extend through the patient's body wall. In a single-lumen drain, the single lumen permits draining fluid from the abdominal cavity, and may optionally permit infusing fluids into the abdominal cavity, and can sometimes be structured to follow a guide installation wire. In a two-lumen drain, the first lumen typically permits draining fluid from the abdominal cavity, and the second may permit infusing fluids into the abdominal cavity, and may also assist in transverse deployment of the field to spread the field over an enhanced area inside the cavity. In a three-lumen drain, or four-lumen drain, the first lumen may permit draining fluid from the abdominal cavity, the second may assist in transverse deployment of the field, and the third lumen may optionally permit infusing fluids into the abdominal cavity in a separate circuit from the first and second lumens, or may communicate to retention structure, such as a balloon. In the four-lumen drain, third lumen may permit infusing fluids into the abdominal cavity in a separate circuit from the other lumen, and the fourth lumen may communicate to retention structure. Embodiments having five, or more, lumen that extend through the patient's body wall are not precluded.

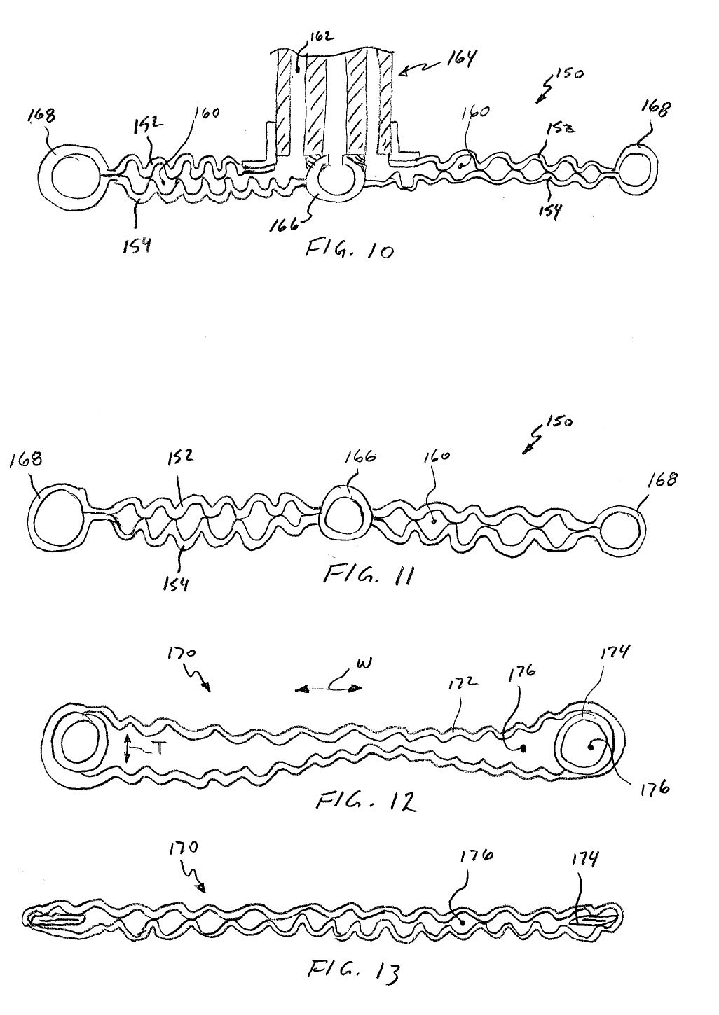

With reference now to FIGS. 6 and 7, the expandable drain field generally indicated at 142 is deployed somewhat like an umbrella. The drain field is first placed into an elongate shape that can slide along an axis through the access opening (and optional access port, if present). Subsequent to passing through the access opening, the cross-section may be deployed transversely with respect to the insertion axis to form a large area that is sometimes desirably disposed between the viscera and the fascia. In the illustrated embodiment 142, internal link elements 144 press the envelope 146 of drain 142 in a direction transverse to deployment shaft 148. It is with contemplation that deployment may be effected by pulling, and/or twisting shaft 148, potentially in combination with manipulation of cooperating actuation structure.

A workable drain field embodiment may have a substantially circular first cross-section (a “stowed” cross-section) sized to fit through the access opening. In such case, deployment may cause circumferential expansion of a cross-section of the drain field. In some cases, the drain field may deploy by some combination of unfolding, unfurling, and/or some other form of expanding to increase a size of the drain field in at least one direction.

One desirable deployed drain area is on the order of about 6 inches (15.2 cm) in diameter. Drain fields having larger, or smaller, drain areas are also workable. It is within contemplation to deploy a drain field having a diameter on the order of about 10 inches (25.4 cm), or even more in certain cases. Drain fields having alternative deployed cross-section shapes, such as rectangular, ovoid, triangular, multi-segmented in perimeter, or irregular, are also within contemplation. A drain field having a drain envelope that deploys substantially as a flat pancake having a diameter of about 200 mm (7.9 inches), can provide an active drain field cross-section of about 31,416 mm² (49 in²). In the case where drain apertures are disposed in a dispersed pattern on both sides of a crenellated pancake, the “active” drain field area is approximately twice the cross-section area. For direct comparison of equivalent installed drain areas, the cylindrical area of a conventional, non-expanding, multi-aperture draining catheter with a 10 mm smooth outside diameter and 200 mm inserted drain field length is, at most, about 6, 283 mm² (9.7 in²), and the “active” drain area of such a device is much, much less than that amount. In contrast, the cross-section area provided by a 10 mm diameter access opening is about 78.7 mm² (0.122 in²).

Deployment of a drain field element may be effected solely by way of a self-bias in a drain envelope, itself. Alternatively, a mechanical linkage system (e.g. like the elements of an umbrella, or plurality of members each having a transversely movable knee joint), may be actuated through various operable mechanisms to flare the drain field in a transverse direction. It is also within contemplation that flaring members may simply be biased along their length, and arranged to cooperate effective to flare a drain field when released from confinement inside an introducer. A workable mechanical linkage system, or flaring system, may operate according to principles of operation of micro-wire devices used in currently available vascular umbrellas or filters for IVC. It is within contemplation that an inflatable skeleton may be employed to effect a similar flaring or spreading actuation. Inflation may be effected with either a gas, such as air, or with fluid, such as saline. Desirably, the deployment mechanism is removable, or sufficiently reversible, to permit eventual withdrawal of a deployed drain field through the access opening.

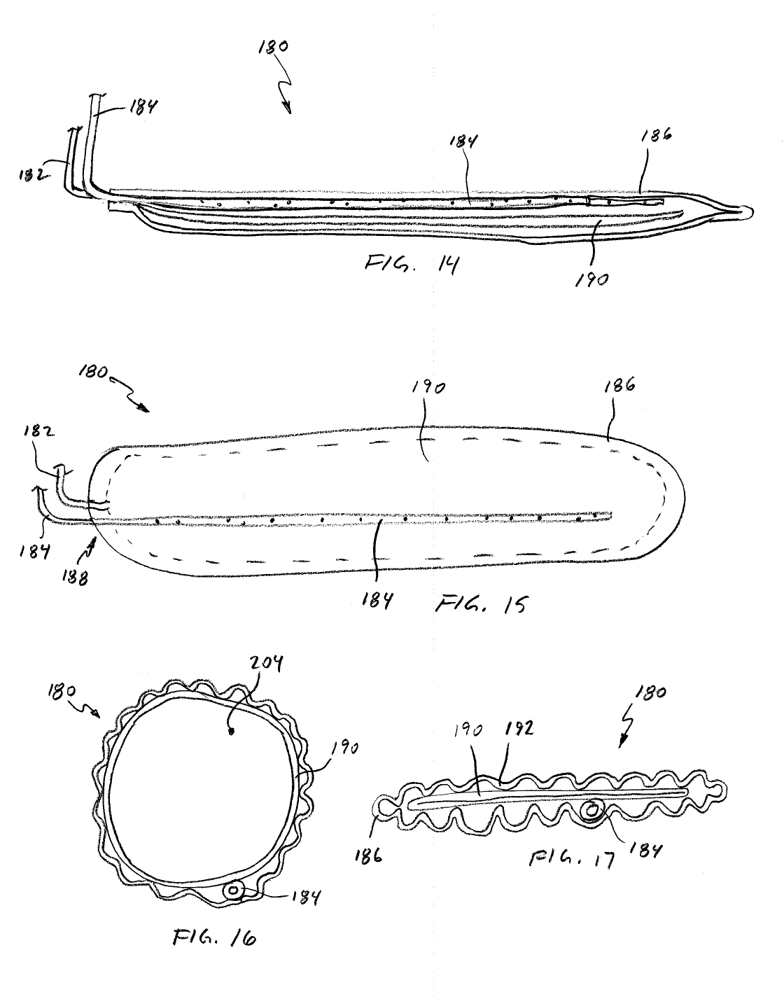

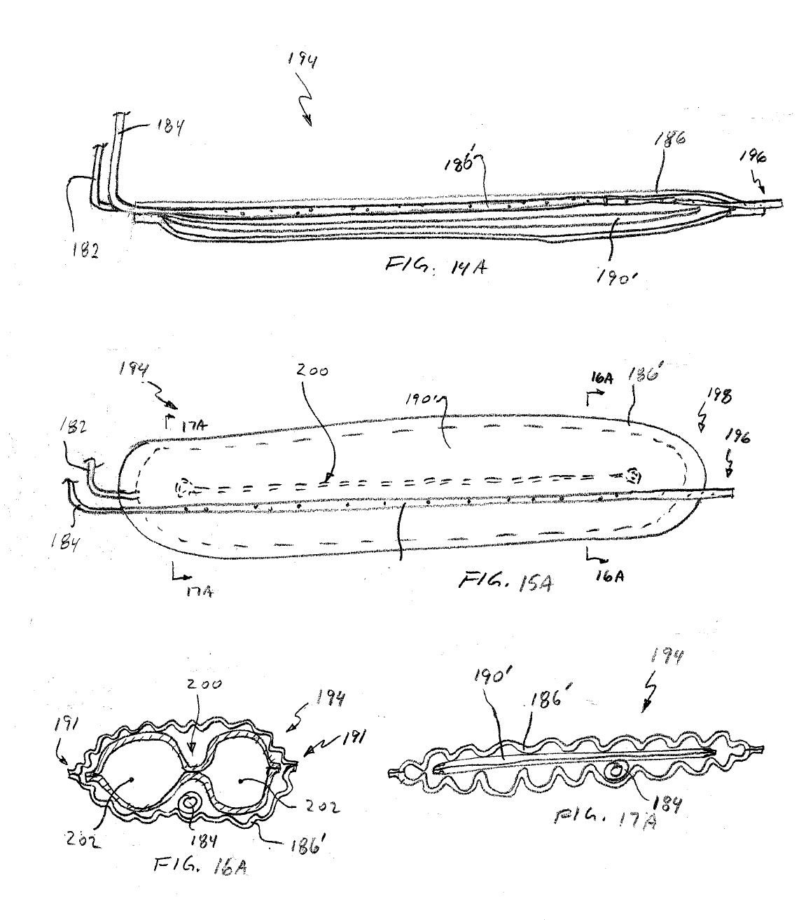

Certain exemplary expandable drain fields are illustrated in FIGS. 8 through 17. With reference to FIG. 8 through 11, portions of a first embodiment, generally 150, may be manufactured from thin membrane material. The membrane may be fused (e.g., RF welded), or otherwise bonded or adhered, together at desired locations to form a pair of oppositely disposed drain envelope membranes, 152, 154, respectively, and an integral inflatable skeleton, generally 156. At least one of the drain envelope membranes is typically perforated by a plurality of small-sized drain apertures, generally 158, and the space 160 between cooperating membranes is in fluid communication with at least one respective drain lumen 162 that extends through multi-lumen conduit 164 for application of suction from exterior the compartment 136. Operable drain apertures 158 may be on the order of about 1 mm in diameter, although larger, or smaller, apertures are also workable.

The embodiment illustrated in FIGS. 8 through 11 includes an integral inflatable skeleton 156 including a centrally disposed backbone 166 and a perimeter member 168 disposed around the perimeter edge of the drain field. It is within contemplation alternatively to also provide for draining access through drain apertures disposed in a direction generally in the plane of a deployed drain field and through the perimeter (e.g. penetrating the circumferentially disposed deployment lumen, and not illustrated).

The drain field assembly 150 in FIG. 8 is constructed to permit rolling, furling, scrunching, or folding, etc., to present a small cross-section for insertion of the drain field into the patient through the access opening. Subsequent to insertion of the drain field 150 into the peritoneal compartment 136, the hydraulic skeleton 156 can be inflated to transversely deploy the drain field. Then, the skeleton members 166, 168 may be deflated. Sometimes, skeleton members of an installed drain field may be temporarily re-inflated, e.g. to improve a drain path for drain fluids, or to readjust the deployment of the drain field 150.

It is within contemplation that one or more skeleton member, or other inflatable structure, may even remain inflated for the duration of deployment of a drain field. For example, and as described in more detail below, it is within contemplation that a drain field may include an inflatable balloon-like compartment that serves as a stopper to resist accidental withdrawal of the drain field from an installed position. Such balloon-like element may function also as a plug to resist undesired, or uncontrolled, escape of fluids from inside the compartment or cavity and through the access opening. Further, inflation, or partial inflation, of certain skeleton members may provide an enhanced path along which fluid may be urged to flow toward a drain lumen of the device.

In certain preferred embodiments, the membrane forming a drain field envelope are crenellated, or otherwise include structure configured to resist complete collapse of the membrane sheets onto each other effective to occlude a drain path for fluid toward the drain lumen. Operable crenellation structure includes wrinkles, spaced-apart posts, pillars, waffle-structure, ribs, arches and dishes, an internally disposed layer of open-celled foam, or any other gap-inducing structure effective to urge presence of at least one continuous drain channel communicating from a plurality of drain apertures toward a drain lumen. Desirably, similar such drain-path enforcement structure may also be disposed on the exterior of the drain membranes, to resist occlusion of a drain path to a plurality of drain apertures by contact between the drain field membrane and the fascia or viscera. Operable crenellation structure may be molded or pressed into the membrane material in a reel-to-reel operation. Apertures may also be formed in such a reel-to-reel operation.

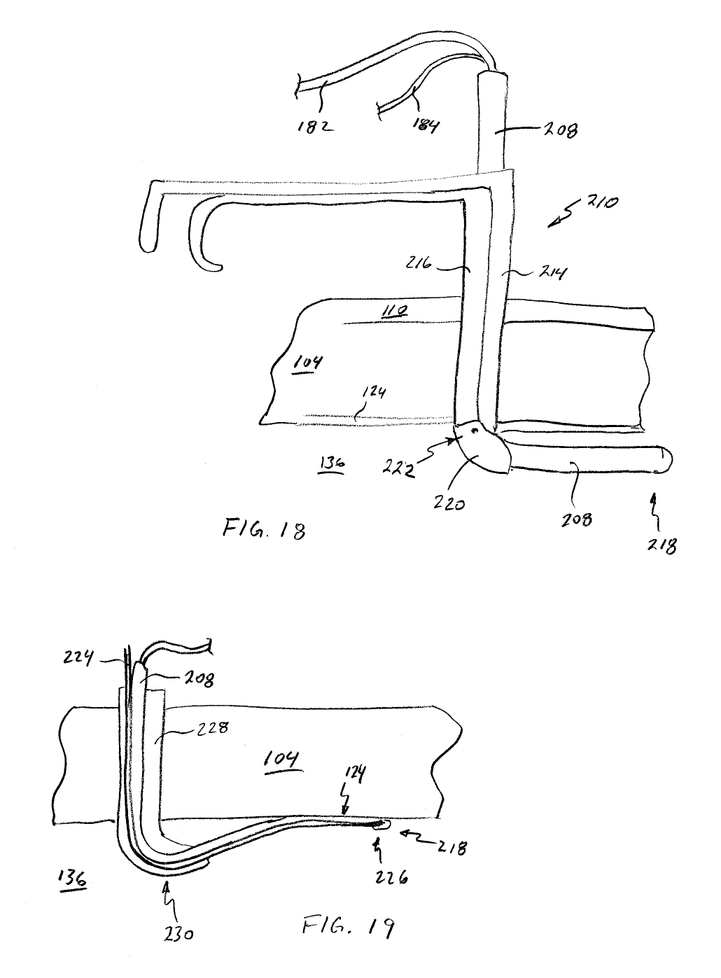

FIGS. 12 and 13 illustrate certain desirable aspects in a hydraulic deployment arrangement for a drain field, generally 170. The drain field 170 may be formed essentially as an envelope 172 by welding a perimeter of a pair of stacked membranes. Sometimes, the perimeter is ovaloid, or rounded, to assist in alignment with principle load-carrying direction capability of a hydraulic skeleton. The hydraulic skeleton member 174 (sometimes called inflation lumen), may be inserted into, or trapped in, space 176 inside envelope 172 during manufacture of, the envelope 172. The drain field can then be rolled, folded, or otherwise compressed to have a small cross-section that will fit through the access opening of an access tunnel having a desirably small size.

After the drain field is inserted into the patient's abdominal compartment, the inflation lumen may be pressurized to expand space 178 and deploy the drain field envelope to a desired deployed configuration, e.g. increasing in width “W,” as illustrated in FIG. 12. Subsequently, the skeleton member may be deflated during use of the drain field to remove fluid from the compartment, as indicated in FIG. 13. However, such deflation is not always required during use of the drain field. A deflatable skeleton does, however, desirably permit the drain field to be removed from the compartment through the access opening.

A pressurized skeleton member may cause a transverse, or thickness direction, separation between the top and bottom membranes of an envelope, as indicated at “T” in FIG. 12. Such separation may sometimes be used to advantage to re-open clogged fluid flow paths that extend between drain apertures in the membranes and a drain conduit, or are between the drain field and viscera or fascia. Such clogging may result from blood clotting, protein deposits, or build-up of other material. Re-pressurizing a hydraulic skeleton may also advantageously re-deploy a drain field that might have not initially fully deployed, or that has shifted responsive to patient movements.

FIGS. 14 through 17 illustrate another currently preferred expandable drain field, generally indicated at 180. While the illustrated drain field 180 is substantially ovaloid and elongate in an insertion direction, it is within contemplation that the drain field may be formed in other shapes. The illustrated elongate shape advantageously permits insertion of a larger total drain area through a small-sized access opening, and facilitates compressing the drain field to a minimum stowed diameter extending in-line with a drain conduit umbilical.

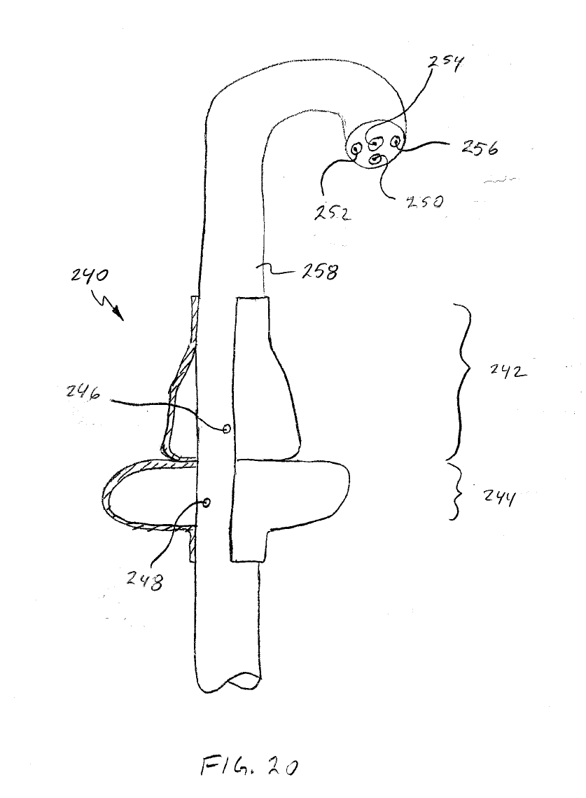

As illustrated in FIGS. 14 and 15, the inflation conduit 182 and drain conduit 184 can enter the drain field envelope 186 at a side of a smaller edge, generally 188, which may facilitate removal of the deployed drain field 180 through an access opening having a desirably small size. The portion of drain conduit 184 disposed inside the drain field 180 may be perforated, as illustrated. Once inserted through an access opening, hydraulic skeleton member 190 may be inflated, as illustrated in FIG. 16, to spread out the drain field envelope 186 inside a compartment 136. Typically, envelope 186 is made from a fairly thin and perforated membrane 192. As illustrated, membrane 192 is also crenellated. Therefore, when skeleton member 190 is deflated, as illustrated in FIG. 17, envelope 186 defines void spaces that permit fluid to migrate toward drain conduit 184 and out of compartment 136.

Still with reference to FIGS. 14-17, it is also contemplated that certain embodiments may additionally include a third conduit through which to infuse fluid into the compartment. As further detailed below, a lumen may communicate to inflatable retention and/or sealing structure, such as a balloon. It is contemplated that a plurality of such drain fields may be deployed in a compartment; e.g. to form a circumferentially spaced-apart spoke arrangement; and even through a single access opening. A plurality of such drain fields may advantageously be deployed one-at-a-time, to reduce the required deployment volume that must be accommodated by the patient. Further, sections of deployment skeletal structure of a single drain field may be pressurized and deflated, or otherwise actuated or mechanically deployed, in sequence.

With reference to FIGS. 14A through 17A, it is within contemplation in an alternative construction, generally indicated at 194, that the distal end 196 of the drain conduit 184 may extend completely through the illustrated distal end 198 of the perforated envelope 186. In such case, a guide wire, such as is provided by the Seldinger technique, may be used to guide the furled, or otherwise reduced-in-size, drain field assembly during its insertion through a body wall 104 and into a compartment 136. It is not required that the protruding drain conduit be secured to the perforated envelope at the distal penetration site, although such would sometimes assist in manipulating the drain envelope 186, e.g., facilitating use of the drain conduit 184 as a tool to position the distal end of the envelope 186 during insertion of the drain field 194.

With particular reference to FIGS. 15A and 16A, it is within contemplation to provide an alternatively structured internal skeleton member 190′ that is configured to reduce a total amount of fluid required to cause a deployed shape. A preferred alternative skeleton structure 190′ also urges the deployed drain envelope 186 toward a preferred, somewhat planar, orientation. It should be noted that the drain envelope 186 in FIG. 16A provides substantially the same circumferential length as the alternative envelope 186′ illustrated in FIG. 16. Alternative envelope 186′, as illustrated in FIG. 16A, may be formed by bonding perimeter edges, generally indicated at 191 of top and bottom membranes.

As further illustrated in FIG. 16A, an operable skeleton member 190′ may be formed by adhering opposite sides of the illustrated inflation balloon structure together along one or more dividing line, or seam, generally 200, stretching along only a portion of the skeleton member's length axis. Such construction essentially forms a plurality of parallel inflatable channels, each such channel having a smaller internal space 202 than the space 204 inside of cross-section illustrated in FIG. 16, and consequently the assembly requires a correspondingly smaller amount of fluid to deploy. When inflated, the parallel channels also inherently urge the drain field envelope to expand in a more planar shape, compared to the substantially round cross-section illustrated in FIG. 16.

Deployment of an exemplary embodiment 208, structured similarly to the embodiment illustrated in FIGS. 14 through 17, will now be described with reference now to FIG. 18. An installation-assisting tool, generally 210, may be used to facilitate the procedure. Certain embodiments of the tool 210, and as illustrated, can sometimes be used to stretch the access opening, or at least a portion of the access tunnel through the body wall, by a certain amount. Opposite pushing and pulling jaws 214, 216, respectively, may be displaced apart to enlarge a space in the hollow core there-between. The compressed, compacted, furled, scrunched, or otherwise reduced-in-cross-section size exemplary drain field 208 is then inserted through the hollow core provided by the tool 210 and into the compartment 136. However, it is within contemplation that a drain field structured in accordance with certain principles of the invention may be configured for insertion through an access opening without requiring either aid of an insertion assisting device, or access port structure.

Also as illustrated in FIG. 18, an insertion-assisting tool 210 may be structured to assist in orienting the advancing distal tip 218 of the compressed drain field 208 to place the inserted drain field in a desired orientation inside the compartment. For example, a simple linkage system (not illustrated) may be operated to cause the distal guide surface 220 to rotate around a pivot point, generally 222, effective to deflect the advancing drain field and cause the tip 218 of the drain field to progress in a substantially transverse direction, desirably placing the deployed drain field 208 between the viscera and fascia 124. However, it is also believed to be acceptable for certain embodiments of a drain field to be inserted in a substantially straight path extending transverse to the fascia 124 and into the cavity 136 or compartment, where deployment of the drain field 208 will be accommodated by movement of the internal organs, or guts.

An exemplary embodiment, such as illustrated in FIGS. 14 through 17 and 14A through 17A, may be furled, folded, or otherwise compacted around the perforated drain conduit portion to form a profile that can be inserted through the opening afforded by the access port or unaugmented access opening. The drain conduit 184 may then act somewhat as a spine to urge the compacted drain along a desired (e.g. substantially straight) insertion path. An operable drain conduit 184 may alternatively, or additionally, be self-biased and oriented during the installation procedure effective to guide the distal end in a preferred direction. It is within contemplation for tracking structure (such as radio-opaque structure), to be included to permit substantially real-time tracking during an installation process, or to verify effective deployment of an installed drain field. A magnetically attracted distal tip 218 can be guided by application of an appropriate magnetic field during installation of a drain field.

Certain drain field envelopes and/or inflation members and/or suction or inflation conduits, may be configured to provide axial stiffness, on their own, sufficient to guide the distal end during installation. For certain other embodiments, once the proximal end of the drain reaches the tool or skin surface, a plunger 224, e.g., a finger, or dedicated tool, may be used to further advance the proximal end of the drain field through the body wall. For other embodiments, the suction conduit 182 (alone, or in combination with one or more other conduit) portion extending proximal the drain field may provide sufficient axial stiffness to permit its use to incrementally advance the drain field to an installed position.

With reference now to FIG. 19, a self-biased installation aid 224 may be used to drag certain embodiments of drain fields toward a deployed position. The illustrated aid 224 is essentially embodied as an elongate spatula, which is normally disposed in an arcuate configuration. The spatula blade tip 226 is inserted into a pocket provided at the distal end 218 of the drain field, and drags the drain along as the spatula tip 226 is advanced. The blunt tip and arcuate axial shape of the spatula urges the tip to follow the fascia 124, and facilitate deployment of the drain between the viscera and fascia. Subsequent to insertion to an installed length, the spatula 224 is retracted from the pocket, leaving the drain field behind. As illustrated in FIG. 19, an insertion aid 224 may sometimes be further guided by a tool, such as an installation tool 228 or access port having a distal guide surface 230 effective to orient the distal end of a drain field. The installed drain field may sometimes be further deployed, such as by pressurizing a deployment member, to increase an effective size of the installed drain field area.

It is known to include an inflatable balloon near the distal tip of certain urinary catheters. Such catheters typically include at least one drain opening disposed distal to the balloon (by quite a bit less than about 3 inches). In any case, the “active” drain field of such catheters (which may include only one or two apertures), disposed distal to the balloon is considerably less than 1 in². The inflated balloon is effective to resist undesired withdrawal of the catheter from an installed position with respect to a bladder. The actual fluid seal to resist leaking of urine from the bladder is provided by the patient's urinary sphincter, which clamps in fluid-resistant engagement against a mid-span portion of the catheter.

It is desirable to include an inflatable balloon in certain embodiments of drain fields structured according to certain principles of the instant invention. As illustrated in FIG. 20, one embodiment of such inflatable balloon structure, generally 240, includes a plug portion 242 and a stopper portion 244. The illustrated plug portion 242 is configured to cooperate with structure associated with the interior aperture of the tunnel of a patient's access opening 102. The illustrated plug 242 is also capable of inflation (by way of inflation aperture 246) to a size that is larger than the internal aperture of the access tunnel 102. A fluid resistant seal can therefore be formed when the plug portion 242 is inflated to clamp against the aperture and/or part of the access tunnel 102. An inflated stopper 244 can sometimes also be drawn into engagement with the internal aperture (even without a plug portion 242) to form a leak-resistant seal and alternative plug. In the latter case, tension is typically maintained on the proximal portion of the catheter to urge formation of a fluid-resistant seal between the stopper 244 and internal wall of the compartment 136, or the internal aperture of access tunnel 102.

As further illustrated in FIG. 20, stopper 244 communicates through inflation aperture 248 to stopper lumen 250 to permit stopper inflation. Similarly, plug 242 communicates through inflation aperture 246 to stopper lumen 250 to permit stopper inflation. Additional lumen 254, 256 provided by multi-lumen conduit 258 are dedicated to drain discharge and deployment/expansion of the drain field, respectively.

An inflated stopper portion is desirably larger than the interior aperture of an access tunnel 102, and will therefore cause an interference to resist undesired removal of the installed drain field. As illustrated, the stopper portion 244 and the plug portion 242 may be structured to permit individual and separate inflation. In an alternative operable arrangement, the stopper portion and plug portion may be arranged for joint inflation. When structured for separate inflation, the stopper 244 is typically inflated first, and pulled into contact with the fascia. Such procedure may automatically place the stopper portion 244 into proper location to cooperate with the internal aperture and/or portion of the access tunnel 102. Then, the plug portion 242 may be inflated to form a fluid resistant seal. Sometimes, the plug portion 242 may be visualized during the procedure, and provide feedback regarding position and location of the stopper 244 and/or plug 242 structure(s). In any case, the stopper 244 (and plug 242, if present) can be deflated to permit removal of the drain field through the access opening.

It is generally desirable to limit the amount of fluid or volume required to deploy a drain field, to reduce imparting excessive pressure inside the compartment 136 as a consequence of deployment. Therefore, it is within contemplation that a kit may be provided to include a dispensing device that is pre-loaded to administer only a known amount of deployment volume.

A typical installation procedure may be considered as requiring only a small extension to the well-known Seldinger technique. One exemplary installation procedure includes the following steps: 1) puncture skin, fascia, and abdominal peritoneum with a large bore needle; 2) thread wire through needle bore and into cavity using Seldinger technique; 3) remove needle; 4) incise skin approximately 10-12 mm around wire; 5) dilate fat and fascia over the wire using sequentially larger dilators to form an access hole; 6) thread drain assembly onto the wire (sometimes with either an internal guide, or an external peel-away or slide-off guide, such guides may be substantially rigid); 7) push distal end of drain assembly through access hole until the drain field is within the abdominal/peritoneal compartment; 8) remove guide (if present) and remove wire; 9) inflate retention balloon/inflation skeleton using supplied sterile saline; 10) tug gently on proximally protruding conduit segment to ensure drain field is in place with retention structure against the internal abdominal wall; 11) slide external retention clamp down conduit segment and to skin surface, then gently tighten clamp against proximal conduit, and suture clamp to skin (an optional second clamp affixed to the conduit and several inches proximal the first clamp may also be sutured to the skin to provide redundant defense against inadvertent removal of the drain field); and 12) attach drain suction lumen to a suction source, or effluent container for gravity extraction.

With reference now to FIG. 21, an operable tool to assist in insertion of a drain field may include an internally disposed introducer 260, or even a dilator of sufficiently small diameter. As illustrated, introducer 260 is structured to follow wire 262 into the compartment 136 and bring with it the deployable donut 264 and dual-lumen catheter 266.

FIG. 22 illustrates an alternative externally disposed introducer, generally 270, that may be employed to resist premature displacement of the compacted drain field from a stowed position during the insertion procedure. Such an external introducer 270 may sometimes be configured to separate into clamshell portions to facilitate axial motion, of the shell portions relative to the drain field, to facilitate extraction of the introducer parts. In one embodiment of an external introducer, a shear force may be imparted effective to separate clamshell portions along an axis that is directed in an insertion direction, or length axis of the introducer.

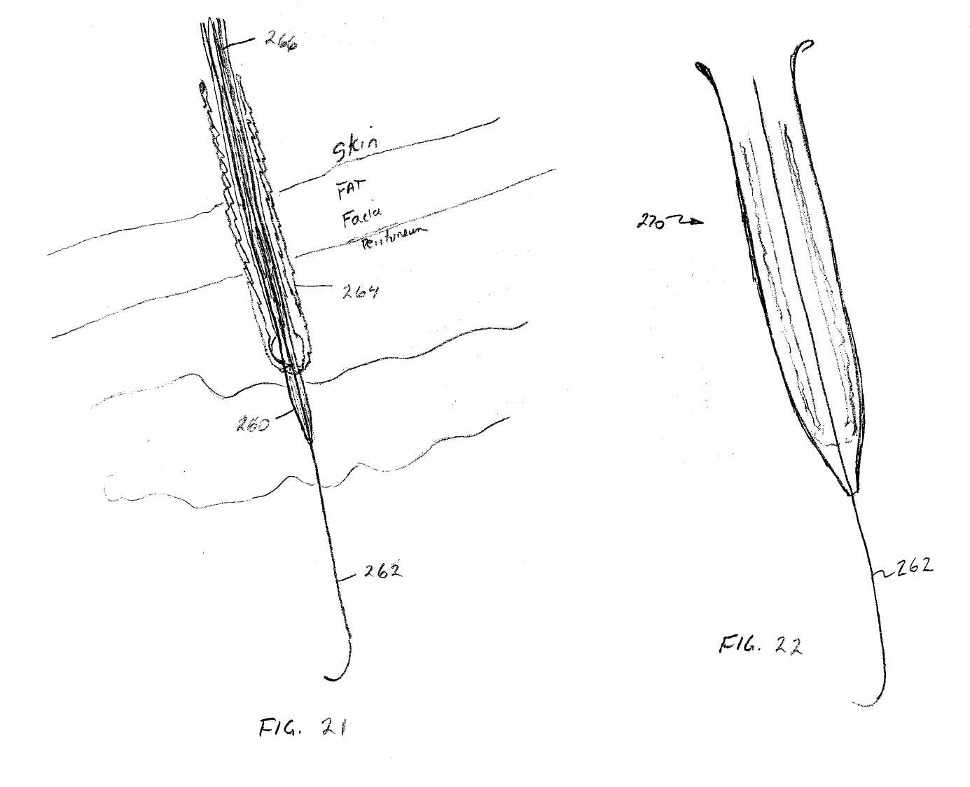

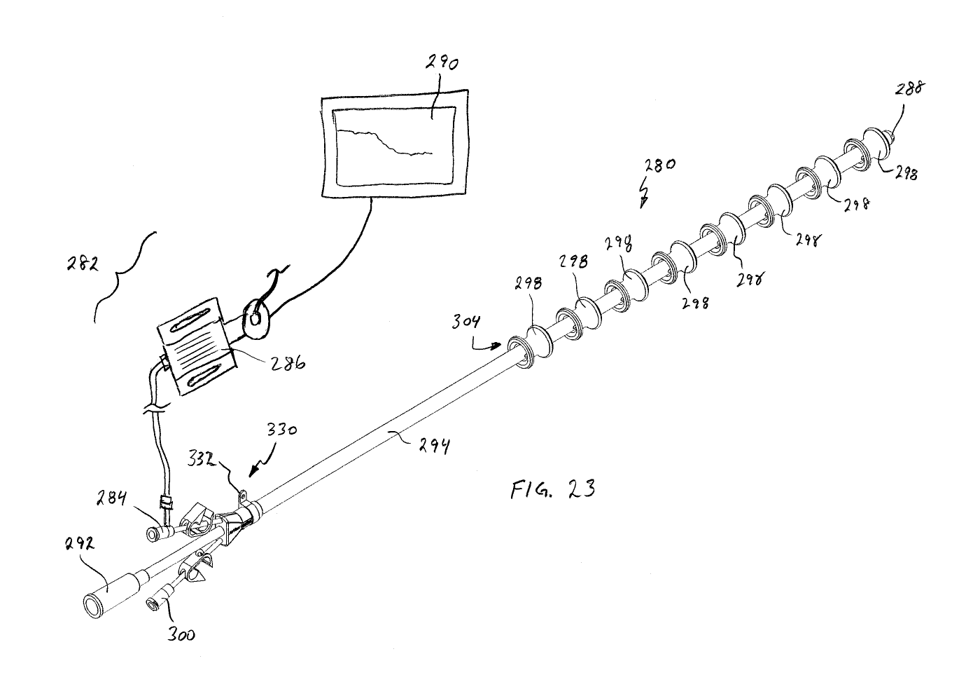

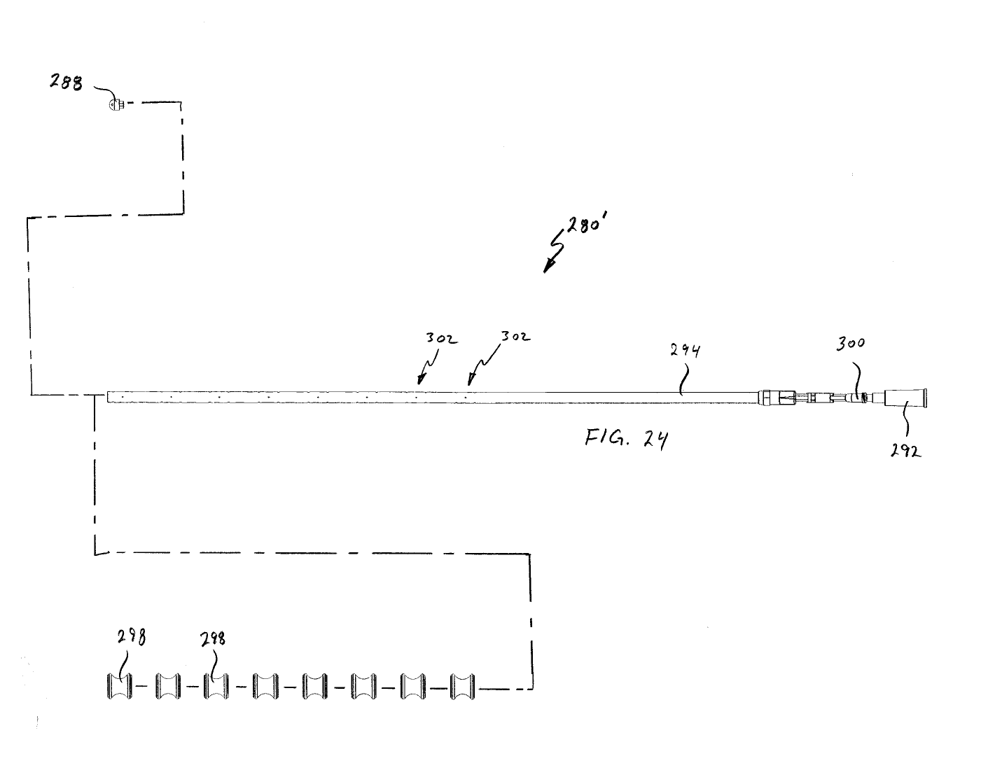

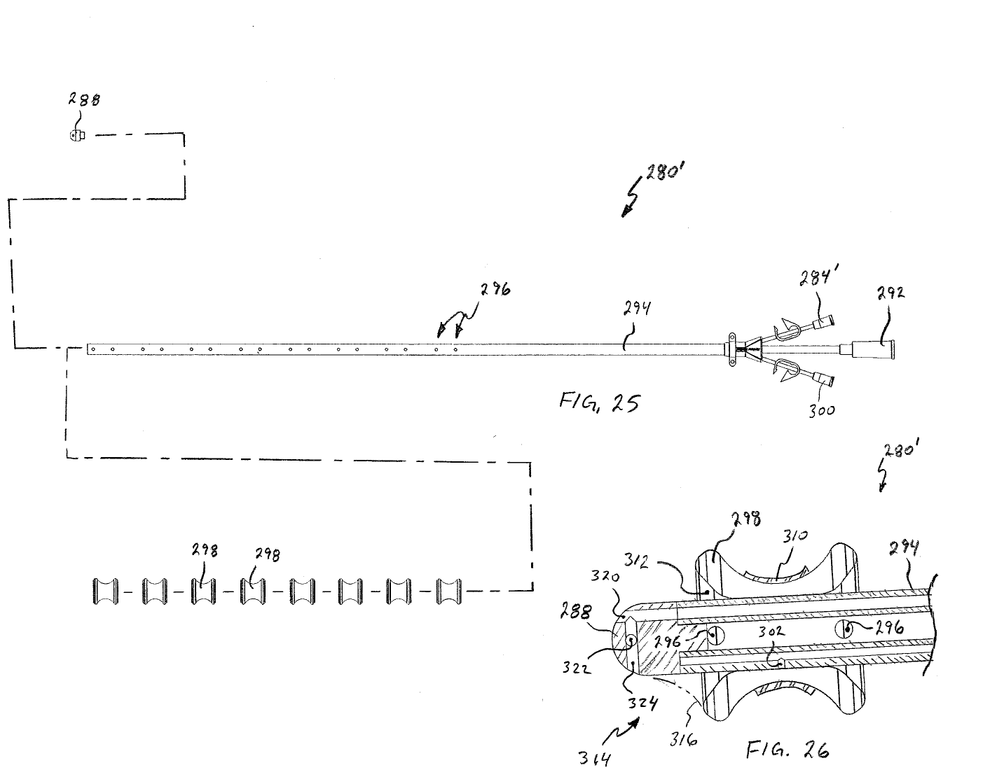

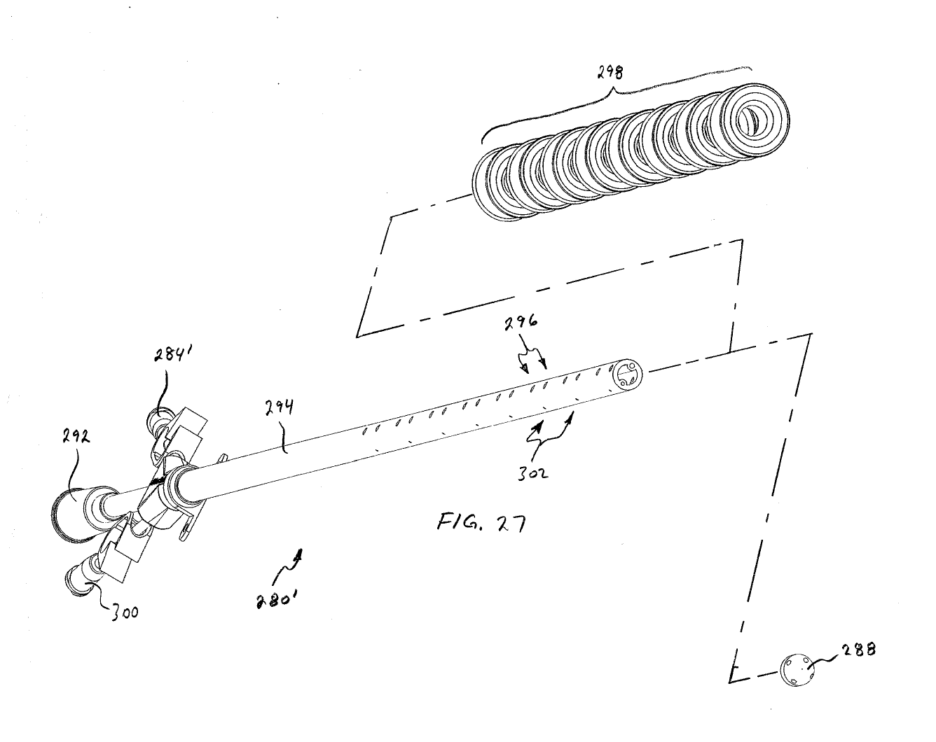

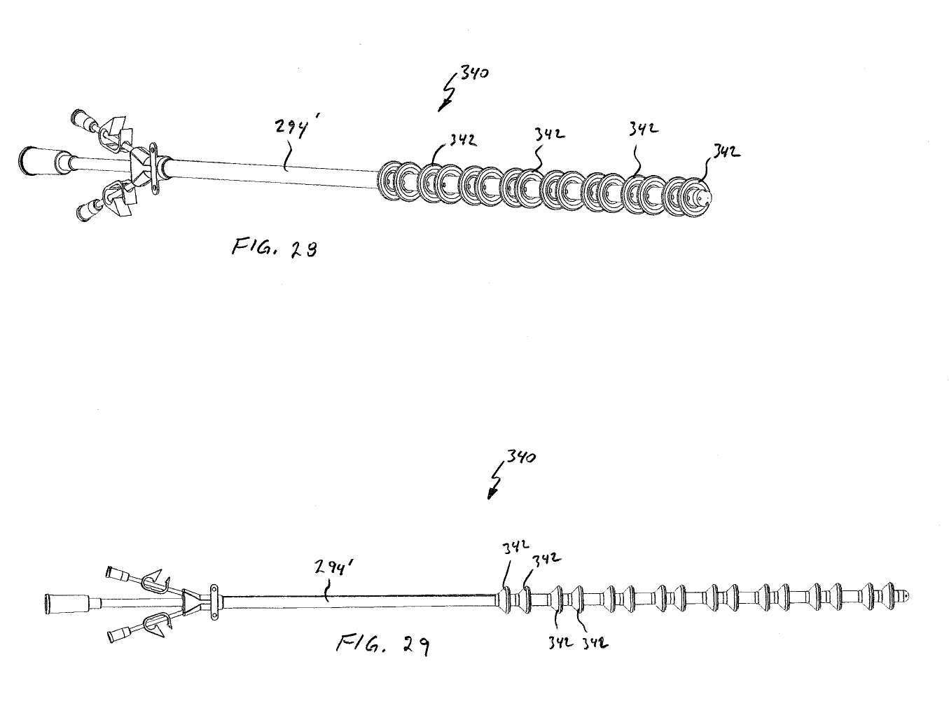

Certain details of construction of a currently preferred abdominal catheter, generally 280, are illustrated in FIGS. 23 through 27. FIG. 23 shows catheter 280 incorporated in an assembly, generally 282, that is also operable to track intra-abdominal pressure. Infusion connector 284 in FIG. 23 is adapted as a three-way hub to permit fluid communication with pressure transducer 286, as well as through distal tip 288 of catheter 280. Therefore, real-time intra-abdominal pressure may be monitored when catheter 280 is deployed in a medical patient. The catheter embodiment 280′ in FIGS. 24 through 27 include a conventional connector 284′. However, a conventional three-way conduit connector may be added to provide the same pressure measurement functionality. A visual display device 290 may be used to indicate an instantaneous pressure value or trend, such as by way of a plot of the measured pressure over a time interval.

A drain connector 292 is in fluid communication through catheter body 294 to a plurality of drain apertures 296 (see FIG. 26) to permit collection of fluid from a compartment 136. A plurality of inflatable balloons 298 are illustrated as being individually disposed to act as bridge elements to protect individual drain apertures 296 from tissue infiltration during a prolonged dwell interval. Inflation connection 300 is in fluid communication with the interior of each such balloon by way of inflation apertures 302 (see FIG. 26).

Prior to deployment in a patient's compartment 136, balloons 298 are deflated and occupy minimum radial space in excess of the diameter of body 294 to facilitate insertion of distal tip through an access tunnel 102. Illustrated body 294 is about 18 inches (46 cm) in length, and has about 8 inches (20 cm) of extension length proximal to the most proximal balloon 298. Once a desired insertion length is effected, balloons 298 are inflated to move viscera away from drain apertures 296. Inflation may be accomplished by way of fluid or gas. With reference to FIG. 23, and if desired, the body 294 may then be gently refracted to place proximal end 304 of a proximal balloon 298 into play as a locating stopper, and possibly as a seal element for the interior opening of an access tunnel 102. Of course, a dedicated plug element 242, or stopper element 244, can be provided in an alternative embodiment within contemplation.

With reference now to FIG. 26, it is currently preferred that part of a balloon 298 be structured to act as a bridge element for a cooperating drain aperture 296. As previously mentioned, a bridge increases the active area associated with an aperture. The balloon 298 illustrated in FIG. 26 is reinforced by band element 310 to enforce a generalized dogbone shape. Such a shape produces a space 312 disposed at proximal and distal ends of the balloon 298, each such space 312 resembling a space inside the bell of a trumpet. The catheter body 294 penetrates space 312, and reduces an effective opening size of the cross-section area of the space 312.

An active area can be relied-upon to apply a suction over that area to the viscera. The term minimum active area may be defined as the unoccludable area associated with an aperture. For a conventional smooth-walled catheter having a side-hole, an unoccludable active area consists of the area of the side-hole aperture opening. An effective bridge element increases the minimum unoccludable area. In the case of one bridge/aperture zone, generally indicated at 314 in FIG. 26, the minimum unoccludable area is an annular area formed by the inflated and axially open end of a balloon 298, minus the cross-section area of the catheter body 294. The minimum active area is generally a conservative estimate of active area. It is believed that a realistic active area at a zone 314 is larger than the minimum active area, because viscera will tend to form a generally catenary “hypotenuse” (indicated by phantom line 316 in FIG. 26), rather than a perpendicular leg between the surface of body 294 and a rim of a balloon 298.

In the non-limiting case of the exemplary embodiment 280′ illustrated in FIGS. 25 and 26, the diameter of an axially open end of a balloon 298 (e.g. a minimum-spacing circle defined as being disposed in a plane that is perpendicular to a length axis of catheter body 294 and tangent to the axially open bell-shape defined by an illustrated balloon 298), is 0.6 inch (15.2 mm). For completeness of disclosure, the maximum diameter of an illustrated balloon 298 is about 0.75 inch (19 mm). The diameter of illustrated catheter body 294 is 0.315 inch (8 mm). The diameter of a drain aperture 296 is 0.08 inch (2.0 mm). The diameter of a largest preferred access tunnel is about 0.394 inch (10 mm), so a preferred maximum tunnel cross-section area is about 0.122 inch² (78.7 mm²). Conservatively, illustrated balloon 298 produces a minimum active area of 0.2048 inch² (132.1 mm²) for each zone 314. Therefore, a total minimum active area for illustrated catheter 280′ having 16 zones 314 is 3.28 inches² (2,114 mm²). If the bridge elements provided by balloons 298 were not included, the 32 apertures 296 would produce an active area of only 0.161 inch² (103.8 mm²).

Therefore, it may be observed by inspection that certain embodiments constructed according to certain principles of the instant invention may be deployed to cause a conservative active drain area that is about an order of magnitude greater than the cross-section area of an insertion access tunnel required to place the catheter inside a medical patient. The number of inflation elements may be varied, so the active drain area provided by certain embodiments can be further increased, or decreased, as desired. Further, bridge elements may be structured such that their inflated size is larger, or smaller, than the numerical example immediately above, to cause a corresponding change in deployed active drain area.

With reference to FIG. 26, illustrated drain apertures 296 are both disposed inside a volume provided by space 312, and are disposed axially interior to a hypothetical cap formed by the minimum-spacing circle. It is preferable for a drain aperture to be disposed such that viscera will not infiltrate the drain aperture while an associated bridge element is deployed. However, it is believed that a drain aperture including a portion disposed axially anywhere inside a cap, formed by sweeping curve 316 about a local length axis of catheter body 294, is effective to impose the enhanced active drain area, associated with a bridge/aperture pair, onto the viscera.

Still with reference to FIG. 26, tip 288 includes lumen 320 in fluid communication with infusion connector 284 to permit infusing fluids into the abdominal compartment. Lumen 320 also may serve as a passage for a guide wire to facilitate installation of an abdominal catheter. Sometimes, it is desirable for an abdominal catheter to spray infusion fluid in a plurality of directions. As illustrated, a workable tip 288 may also include a cross-axis lumen 322, which communicates fluid in, and/or out, of the plane of the drawing sheet. A transverse lumen 324 may be provided to communicate infusion fluid in an additional direction from tip 288.

With reference now to FIG. 23, it is desirable to provide one or more suture wing, generally indicated at 330. A suture wing provides structure to facilitate holding a portion of a catheter in registration with a patient. Illustrated wing 330 is free to rotate about the local length axis of catheter 280, but is trapped to resist axial motion there-along. Typically, a surgeon may stitch one or more suture through a hole in suture foot 332 to attach the flat contact surface of foot 332 to the patient's skin. It is within contemplation to include one or more additional suture wing that may slide axially along the body 294, or even to permit such axial degree of freedom in an alternative wing 330. Such a slidable arrangement may permit affixing an installed catheter to skin in the immediate vicinity of the access tunnel. An alternative configuration of a suture wing 330 provides additional suture-holding foot structure, oriented at 90-degrees with respect to illustrated foot 332 and configured for approach in a distal direction to contact with skin. Such an arrangement may be sutured as desired to hold the local catheter length axis either substantially parallel to the skin, or perpendicular to the skin, as appropriate in a given circumstance. Structure to hold a catheter in registration with a patient at other intermediate angles is also within contemplation.

FIGS. 28 and 29 illustrate an alternatively structured abdominal catheter, generally 340. Catheter 340 is structured substantially similar to catheters 280 and 280′, except the inflatable bridge elements 342 carried by catheter 340 are adapted to facilitate removal from the patient. A proximal end of each element 342 is affixed in close agreement with the surface of the catheter body 294′, and forms an automatic wedge-shape to facilitate guiding the proximal end of a deflated balloon element 342 into, and through, the access tunnel. Of course, each inflatable element 342 is in communication through its associated inflation aperture to an inflation source.

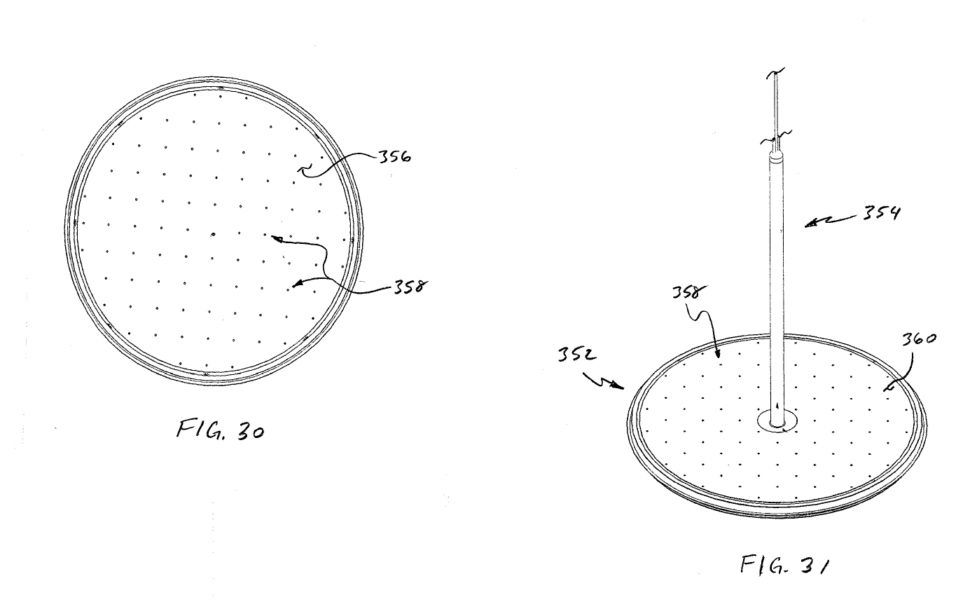

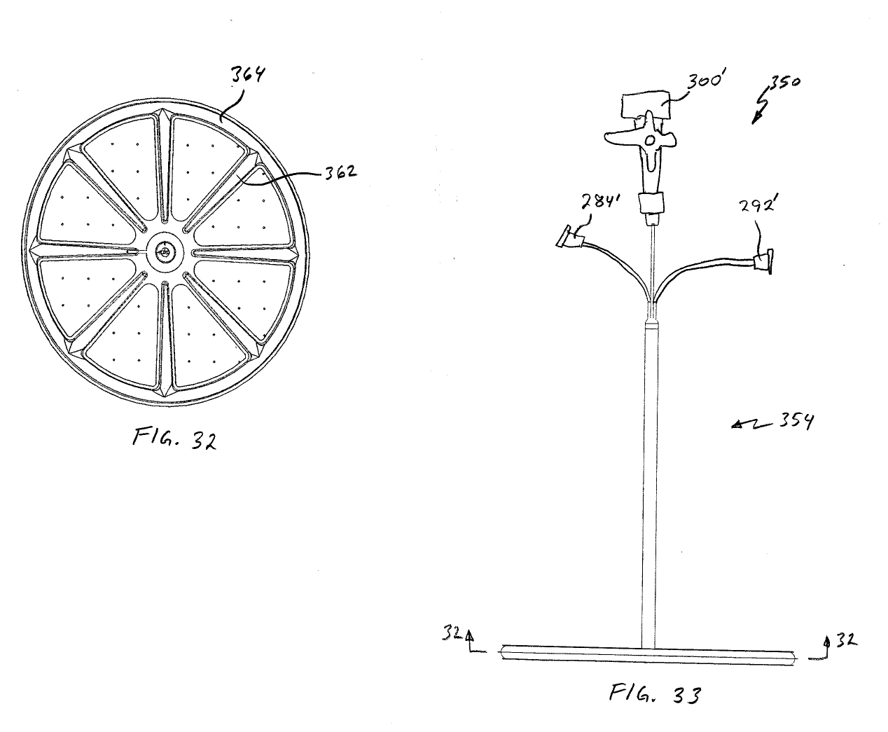

FIGS. 30 through 34 illustrate details of construction of another operable abdominal catheter, generally 350 in FIG. 33. Catheter 350 may be characterized as a pancake-shaped drain field, generally 352, having a centrally disposed umbilical, generally 354. The umbilical 354 desirably has a length at least sufficient to extend through a body wall of a medical patient when deploying the drain field 352. Typically, the diameter of the umbilical 354 is sufficiently small as to permit furling, scrunching, folding, or otherwise compacting the drain field in a stowed position surrounding the umbilical and permit insertion of the stowed assembly through an access tunnel of a desired small cross-section size.

A distal surface 356 of drain field 352 carries a plurality of drain apertures, generally 358. Proximal surface 360 also carries a plurality of apertures 358. A volume disposed between distal surface 356 and proximal surface 360 forms a lumen through which fluid extracted from an abdominal compartment may communicate through drain lumen 162 toward a drain receptacle.

Apertures 358 on the non-limiting exemplary illustrated embodiment 350 each have a diameter of about 0.031 inch (0.8 mm), and a corresponding total open aperture area (including both sides of the drain field 352) of about 0.1244 in² (80.3 mm²). The diameter of the drain field 352 is about 5.8 inches (146 mm), resulting in a combined proximal and distal drain field area of about 51.9 in² (33,506 mm²). In the case where the proximal and distal surfaces are sufficiently crenellated, the entirety of such combined drain field area may be regarded as being active.

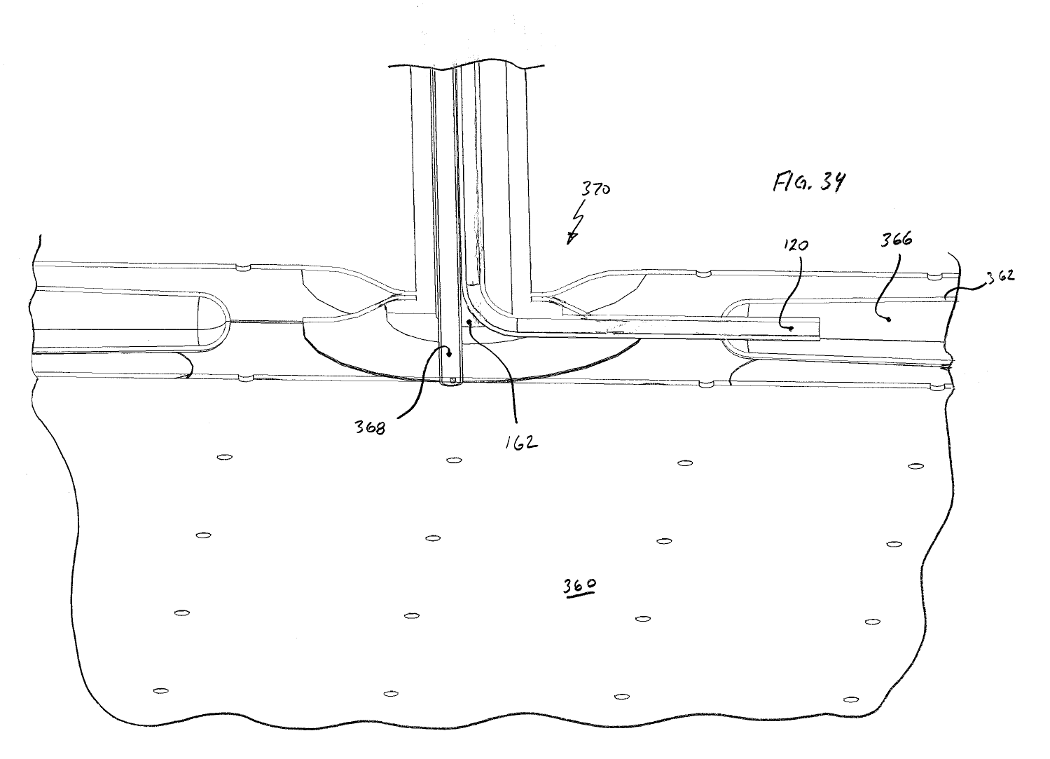

As illustrated in FIG. 32, a plurality of inflatable spokes 362 are disposed in fluid communication with each other through inflatable rim 364. With reference to FIG. 34, inflation lumen 120 communicates to a space 366 inside a spoke 362. A stopcock can be included in the inflation connector 300′ (see FIG. 33), as a means to resist undesired flow of inflation fluid or gas to or from an inflatable element. Infusion lumen 368 communicates from infusion connector 284′ through distal surface 356 to permit infusing fluid into an abdominal compartment, and may also receive a guide wire during installation of a catheter 350. Drain lumen 162 communicates through drain connector 292′, and is sufficiently large in size as to avoid occlusion at the hub area, generally 370, by transversely routing inflation lumen 120 toward a spoke 362.

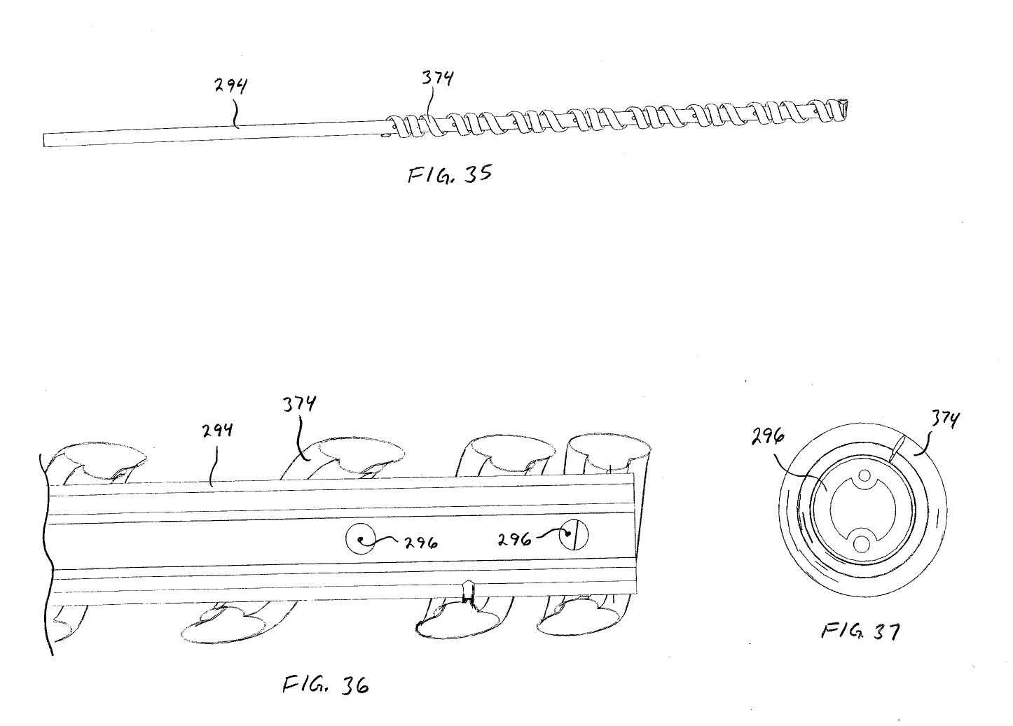

With reference now to FIGS. 35 through 37, it is within contemplation alternatively to provide a catheter body 294 with one or more extended-length, axially extending, or spiral, balloon element 374. Preferably, such balloon element 374 is configured as an axially extending bridge element deployable in harmony with one or more aperture 296. A workable bridge element may resemble a mushroom in cross-section, with the mushroom cap being configured to cause a drooping shape in viscera that disposes at least a portion of a drain aperture inside a protected space at the mushroom stem. Such an extended-length, axially extending, or spiral element may assist in extraction of a deployed catheter, similar to a tapered balloon element 342. That is, the extended length of a balloon can provide a consistent cross-section area to a catheter that is extracted through the access tunnel in the patient's body wall. As illustrated, the spiral balloon shape may vary in pitch, or balloon axial spacing, along the length axis of a catheter body 294. Alternative spiral balloon configurations within contemplation may have a pitch more like the pitch of rifling in a rifle barrel.

Operable materials of construction for an envelope or inflatable element include medical grade versions of: plastics and plastic-like materials, rubber and rubber-like materials, silicone, and other material that may be formulated into thin-walled structures, membranes, and sheets. Exemplary such materials nonexclusively include PET (Polyethylene terephthalate), Nylon, Nylon elastomers, Polyurethane, Silicone, PTFE, PVC, and Crosslinked Polyethylene. Workable envelopes, or balloons, can be formed from very thin membrane materials, e.g. perhaps 0.001 to 0.004 inch (0.025 to 0.1 mm) in thickness, or even less. A preferred manufacturing method of an envelope or inflatable element includes bonding or fusing membrane layers to form a seal around a perimeter. A similar bonding operation may be employed to form one or more dividing line. A workable bonding operation may employ RF welding, or other manufacturing method known and conventionally employed in the field of medical products.

Introducers, dilators, and other elements may be injection molded from medical grade plastic and plastic-like materials. Certain installation-assisting devices may be manufactured from medical grade plastics and plastic-like materials, or optionally from or include a medical grade metal, such as stainless steel. Catheters and conduit portions of an operable drain field element may be manufactured from one or more section of commercially available medical grade tubing. Operable end connectors for certain tubing elements include conventional connectors of the type typically used in medical products, such as luer-locking devices and barb fittings. Syringes make exemplary pumping devices for inflating certain inflatable elements associated with certain embodiments of the invention.

It is to be understood that any one of the various elements that may be assembled to form an embodiment disclosed in this document may be extracted and assembled in combination with one or more other element from a different embodiment of this disclosure, or with one or more other element from an embodiment known in the field of medical or plumbing devices, to form alternative devices structured according to certain principles of the instant invention.

What is claimed is:

1. An apparatus, comprising:
an expandable drain field, said drain field being structured to collapse to a stowed configuration to permit passage of said drain field in a distal direction through a first cross-section area having a first size, and subsequently to expand to present a deployed drain field comprising an active drain area having a second size that is greater than said first size.

2. The apparatus according to claim 1, further comprising:
an insertion-assist tool with a distal end providing a small cross-section structured for insertion through an aperture made in the abdominal wall and fascia of a medical patient to place a distal opening of said insertion-assist tool inside the abdominal compartment of said medical patient.

3. The apparatus according to claim 1, wherein:
said first cross-section is less than about 100 mm²; and
said second cross-section is greater than about 15,000 mm².

4. The apparatus according to claim 2, wherein:
said insertion-assist tool is structured to permit expanding, subsequent to insertion of said distal end into the abdominal wall of said medical patient, to provide a first cross-section, of a tunnel penetrating at least a portion of said abdominal wall, having a larger size than initially required to receive said distal end.

5. The apparatus according to claim 2, wherein:
said insertion-assist tool is structured to provide a first cross-section that is substantially circular.

6. The apparatus according to claim 2, wherein:
said insertion-assist tool is structured to provide a first cross-section that is oblong.

7. The apparatus according to claim 2, wherein:
a distal portion of said insertion-assist tool is structured as a guide surface effective to orient said drain field for insertion of an axis of said drain field in approximately parallel alignment with said fascia.

8. The apparatus according to claim 1, further comprising:
expansion means operable to expand said drain field from an insertion configuration having an insertion cross-section to a deployed configuration having a deployed cross-section area that is larger than said insertion cross-section area.

9. The apparatus according to claim 8, wherein:
said expansion means comprises a hydraulically actuated conduit network.

10. The apparatus according to claim 8, wherein:
said expansion means comprises a mechanical linkage system.

11. The apparatus according to claim 8, wherein:
said expansion means comprises a self-biased member.

12. The apparatus according to claim 1, wherein:
said drain field comprises a first crenellated contact surface;
a plurality of drain apertures are distributed over said contact surface;
structure defines a plurality of fluid drain paths, each such drain path extending from a drain aperture toward a drain port; and
a drain conduit is disposed in fluid communication with said drain port and operable to remove fluid received from a plurality of said drain apertures.

13. The apparatus according to claim 12, wherein:
said drain field comprises a second crenellated contact surface, said first crenellated contact surface and said second crenellated contact surface being disposed for deployment on opposite sides of said drain field.

14. The apparatus according to claim 12, wherein:
crenellation structure associated with a contact surface also forms a portion of a flow path extending from an aperture toward said drain port.

15. The apparatus according to claim 1, wherein:
said drain field is deployable, from a first shape sized to fit through said first cross-section, to form a comparatively thin and wide structure comprising said second cross-section area.

16. The apparatus according to claim 1, wherein:
said drain field is deployable, from a substantially cylindrical configuration having a first diameter sized to fit through said first cross-section, to form a thin and wide structure having said second cross-section area.

17. The apparatus according to claim 15, wherein:
a perimeter of said second cross-section area is defined by a substantially smooth arcuate path.

18. The apparatus according to claim 15, wherein:
a shape formed by said perimeter of a deployed drain field is substantially circular.

19. The apparatus according to claim 15, wherein:
a shape formed by said perimeter of a deployed drain field is substantially oblong.

20. The apparatus according to claim 15, wherein:
a shape formed by said perimeter of a deployed drain field is substantially irregular.

21. The apparatus according to claim 1, wherein:
said drain field comprises an envelope formed by first and second substantially parallel membranes; and
a skeleton is disposed (in association with)/(internal to) said envelope, said skeleton being configured to urge said drain field toward a deployed configuration.

22. The apparatus according to claim 21, wherein:
at least one of said membranes is crenellated.

23. The apparatus according to claim 21, wherein:
said skeleton may be inflated with a fluid effective to separate a portion of said first membrane from contact with said second membrane.

24. The apparatus according to claim 21, wherein:
said skeleton may be inflated with a fluid effective as part of an installation procedure to deploy a portion of said drain field as a substantially wide and thin object.

25. The apparatus according to claim 21, wherein:
said skeleton comprises a self-biased member structured to urge said drain field toward a deployed configuration.

26. The apparatus according to claim 1, further comprising:
an infusion conduit in fluid communication with at least one infusion aperture to permit infusion of fluid into said compartment.

27. The apparatus according to claim 1, further comprising:
an inflation conduit in fluid communication with a drain-enhancing conduit network, said drain-enhancing conduit network being structured and arranged to permit enlargement of a cross-section of a portion of a drain path effective to establish a fluid flow path through said portion.

28. The apparatus according to claim 1, further comprising:
an insertion tool structured to guide a distal portion of said drain field into an installed position.

29. The apparatus according to claim 28, wherein:
an inserted end of said insertion tool is substantially spatulate in configuration effective to define a space between said fascia and internal organs of said medical patient.

30. The apparatus according to claim 1, wherein:
said drain field is structured to collapse sufficiently to permit withdrawal of a deployed drain field through said first cross-section.

31. An apparatus adapted to provide temporary access through the body wall of a medical patient and into the abdominal cavity of that patient, the apparatus comprising:
a port-tube structured to define an interior passageway there-through between a proximal portion and a distal end, said port-tube carrying an interior flange at said distal end, said interior flange being structured to permit its insertion through an access hole formed in said body wall, and subsequently, to expand to a larger size than a cross section of said access hole, to permit engagement of an area portion of said interior flange against the inside of the peritoneal cavity fascia.

32. The apparatus according to claim 31, wherein:
said interior flange is structured to permit extraction of said interior flange through said access hole subsequent to deployment of said interior flange, inside said patient, to a larger size than a cross-section of said access hole.

33. The apparatus according to claim 32, wherein:
said interior flange is structured to be initially deformed and confined inside a small diameter installer-tube for installation through the body wall and subsequently, biased to deflect into a transversely deployed shape when the installer-tube is withdrawn from the body wall.

34. The apparatus according to claim 33, wherein:
said interior flange comprises inflatable structure effective to enlarge a contact area for engagement of a portion of said contact area against said peritoneal cavity fascia.

35. The apparatus according to claim 33, wherein:
said interior flange is self-biased to urge expansion of said interior flange toward a deployed configuration.

36. The apparatus according to claim 32, wherein:
said interior flange is structured to provide resistance to inadvertent withdrawal of the port-tube.

37. The apparatus according to claim 32, wherein:
said interior flange is structured to form a leak-resistant seal against said peritoneal fascia.

38. The apparatus according to claim 31, further comprising:
an exterior flange with an opening configured for engagement with said port-tube to permit sliding said exterior flange along an axis of said port-tube effective to clamp said body wall between said exterior flange and said interior flange.

39. The apparatus according to claim 32, further comprising:
clamping means effective to maintain an axial position of said exterior flange relative to said port-tube.

40. The apparatus according to claim 39, wherein:
said clamping means comprises cooperating threaded portions of said exterior flange and said port-tube.

41. The apparatus according to claim 39, wherein:
said clamping means comprises one-way ratchet teeth that permit the exterior flange to be shoved toward the interior flange, but resist relative motion in the reverse direction.

42. The apparatus according to claim 31, further comprising:
a fluid-seal associated with the access port effective to resist undesired leakage of fluids from inside said patient and through said port-tube.

43. The apparatus according to claim 42, wherein:
said fluid-seal is structured additionally to seal against one or more tubular structure inserted there-through.

44. The apparatus according to claim 43, wherein:
said fluid-seal comprises a packing material inserted inside said port-tube effective to occlude a cross-section not occupied by said one or more tubular structure.

45. The apparatus according to claim 31, wherein:
said port-tube is structured to operate as an insertion-assist tool through which a drain field may be installed inside said medical patient.

46. In a catheter of the type that is inserted through an orifice, of a tunnel having an interior opening, to dispose a distal end portion of the catheter inside a cavity of a medical patient, the improvement comprising:
an inflatable balloon carried by said catheter at a location proximal to said distal end, certain structure of said inflatable balloon being configured as a plug to form a leak resistant seal in cooperation with structure associated with said interior opening of said tunnel, and certain structure of said inflatable balloon being arranged to form a stopper effective to resist undesired removal of said distal end portion from said cavity.

47. The catheter of claim 46, wherein:
said distal portion comprises a drain field having a deployed drain area in excess of about 1 in² (6.4 cm²).

48. The catheter of claim 46, wherein:
said balloon is disposed proximal to said distal end by a distance in excess of about 3 inches (7.6 cm).

49. An apparatus, comprising:
a drain field comprising a total drain aperture area, said drain field being structured to permit passage of said drain field in a distal direction through a first cross-section area having a first size, and subsequently to present a deployed drain field comprising an active drain area having a size that is greater than said total drain aperture area.

50. The apparatus of claim 49, wherein:
said first size is less than about 0.122 in² (78.7 mm²).

51. The apparatus of claim 49, wherein:
said active drain area comprises an opening formed by a bridge element structured to cooperate with a drain aperture.

52. The apparatus of claim 49, wherein:
said active drain area comprises a crenellated surface structured to cooperate with a drain aperture.
